(19) United States
(12) Patent Application Publication
(10) Pub. No.: US 2021/0380983 A1
(43) Pub. Date: Dec. 9, 2021

(54) ENGINEERING PLANT GENOMES USING CRISPR/Cas SYSTEMS

(71) Applicant: Regents of the University of Minnesota, Minneapolis, MN (US)

(72) Inventors: Daniel F. Voytas, Falcon Heights, MN (US); Paul Atkins, Roseville, MN (US); Nicholas J. Baltes, Maple Grove, MN (US)

(21) Appl. No.: 17/405,577

(22) Filed: Aug. 18, 2021

Related U.S. Application Data
(63) Continuation of application No. 14/629,859, filed on Feb. 24, 2015, now abandoned.
Application No. 14/629,859 is a continuation of application No. 14/211,712, filed on Mar. 14, 2014, now abandoned.
(60) Provisional application No. 61/790,694, filed on Mar. 15, 2013.

(51) Int. Cl. C12N 15/113; C12N 15/82; C12N 9/16; C12N 15/52

(52) U.S. Cl. CPC C12N 15/1131; C12N 15/8203; C12N 15/8205; C12N 15/8213; C12Y 301/21; C12N 15/52; C12N 15/8207; C12N 2750/00043; C12N 9/16

(57) ABSTRACT

Materials and methods for gene targeting using Clustered Regularly Interspersed Short Palindromic Repeats/CRISPR-associated (CRISPR/Cas) systems are provided herein.

### CROSS-REFERENCE TO RELATED APPLICATIONS

This application is a continuation of U.S. Ser. No. 14/629,859, filed on Feb. 24, 2015, which is a continuation of U.S. Ser. No. 14/211,712, filed on Mar. 14, 2014, which claims benefit of priority from U.S. Provisional Application Ser. No. 61/790,694, filed on Mar. 15, 2013.

### STATEMENT AS TO FEDERALLY SPONSORED RESEARCH

This invention was made with government support under DBI0923827 awarded by the National Science Foundation. The government has certain rights in the invention.

### TECHNICAL FIELD

This document relates to materials and methods for gene targeting in plants, and particularly to methods for gene targeting that include using Clustered Regularly Interspersed Short Palindromic Repeats/CRISPR-associated (CRISPR/Cas) systems.

### BACKGROUND

Technologies enabling the precise modification of DNA sequences within living cells can be valuable for both basic and applied research. Precise genome modification—either targeted mutagenesis or gene targeting (GT)—relies on the DNA-repair machinery of the target cell. With respect to targeted mutagenesis, sequence-specific nuclease (SSN)-mediated DNA double-strand breaks (DSBs) are frequently repaired by the error-prone non-homologous end joining (NHEJ) pathway, resulting in mutations at the break site. On the other hand, if a donor molecule is co-delivered with a SSN, the ensuing DSB can stimulate recombination with sequences near the break site with sequences present on the donor molecule. Consequently, any modified sequence carried by the donor molecule will be stably integrated into the genome. Attempts to implement GT in plants often are plagued by extremely low HR frequencies. The majority of the time, donor DNA molecules integrate illegitimately via NHEJ. This process occurs regardless of the size of the homologous “arms,” as increasing the length of homology to approximately 22 kb results in no significant enhancement in GT (Thykjaer et al., Plant Mol Biol, 35:523-530, 1997).

### SUMMARY

This document is based in part on the discovery that the CRISPR/Cas system can be used for plant genome engineering. The CRISPR/Cas system provides a relatively simple, effective tool for generating modifications in genomic DNA at selected sites. CRISPR/Cas systems can be used to create targeted DSBs or single-strand breaks, and can be used for, without limitation, targeted mutagenesis, gene targeting, gene replacement, targeted deletions, targeted inversions, targeted translocations, targeted insertions, and multiplexed genome modification through multiple DSBs in a single cell directed by co-expression of multiple targeting RNAs. This technology can be used to accelerate the rate of functional genetic studies in plants, and to engineer plants with improved characteristics, including enhanced nutritional quality, increased resistance to disease and stress, and heightened production of commercially valuable compounds.

In one aspect, this document features a method for modifying the genomic material in a plant cell. The method can include (a) introducing into the cell a nucleic acid comprising a crRNA and a tracrRNA, or a chimeric cr/tracrRNA hybrid, wherein the crRNA and tracrRNA, or the cr/tracrRNA hybrid, is targeted to a sequence that is endogenous to the plant cell; and (b) introducing into the cell a Cas9 endonuclease molecule that induces a double strand break at or near the sequence to which the crRNA and tracrRNA sequence is targeted, or at or near the sequence to which the cr/tracrRNA hybrid is targeted. The Cas9 endonuclease and the crRNA and tracrRNA, or the tracrRNA hybrid, can be delivered to the plant cell by a DNA virus (e.g., a geminivirus) or an RNA virus (e.g., a tobravirus). The sequences encoding the Cas9 endonuclease and the crRNA and tracrRNA or the cr/tracrRNA can be delivered to the plant cell in a T-DNA, with the delivery being via Agrobacterium or Ensifer. The sequence encoding the Cas9 endonuclease can be operably linked to a promoter that is constitutive, cell specific, inducible, or activated by alternative splicing of a suicide exon. The plant can be monocotyledonous (e.g., wheat, maize, or Setaria), or the plant can be dicotyledonous (e.g., tomato, soybean, tobacco, potato, or Arabidopsis).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

Efficient genome engineering in plants can be enabled by introducing targeted double-strand breaks (DSBs) in a DNA sequence to be modified. These DSBs activate cellular DNA repair pathways, which can be harnessed to achieve desired DNA sequence modifications near the break site. Targeted DSBs can be introduced using sequence-specific nucleases (SSNs), a specialized class of proteins that includes transcription activator-liked (TAL) effector endonucleases, zinc-finger nucleases (ZFNs), and homing endonucleases (HEs). Recognition of a specific DNA sequence is achieved through an interaction with specific amino acids encoded by the SSNs. Prior to the development of TAL effector endonucleases, a challenge of engineering SSNs was the unpredictable context dependencies between amino acids that bind to DNA sequence. While TAL effector endonucleases greatly alleviated this difficulty, their large size (on average, each TAL effector endonuclease monomer contains 2.5-3 kb of coding sequence) and repetitive nature may hinder their use in applications where vector size and stability is a concern (Voytas, Annu Rev Plant Biol, 64, 130301143929006, 2012).

The Clustered Regularly Interspersed Short Palindromic Repeats/CRISPR-associated (CRISPR/Cas) system includes a recently identified type of SSN. CRISPR/Cas molecules are components of a prokaryotic adaptive immune system that is functionally analogous to eukaryotic RNA interference, using RNA base pairing to direct DNA or RNA cleavage. Directing DNA DSBs requires two components: the Cas9 protein, which functions as an endonuclease, and CRISPR RNA (crRNA) and tracer RNA (tracrRNA) sequences that aid in directing the Cas9/RNA complex to target DNA sequence (Makarova et al., Nat Rev Microbiol, 9(6):467-477, 2011). The modification of a single targeting RNA can be sufficient to alter the nucleotide target of a Cas protein. In some cases, crRNA and tracrRNA can be engineered as a single cr/tracrRNA hybrid to direct Cas9 cleavage activity (Jinek et al., Science, 337(6096):816-821, 2012). The CRISPR/Cas system can be used in bacteria, yeast, humans, and zebrafish, as described elsewhere (see, e.g., Jiang et al., Nat Biotechnol, 31(3):233-239, 2013; Dicarlo et al., Nucleic Acids Res, doi:10.1093/nar/gkt135, 2013; Cong et al., Science, 339(6121):819-823, 2013; Mali et al., Science, 339(6121):823-826, 2013; Cho et al., Nat Biotechnol, 31(3):230-232, 2013; and Hwang et al., Nat Biotechnol, 31(3):227-229, 2013). The utility of the CRISPR/Cas system in plants has not previously been demonstrated.

As described herein, CRISPR/Cas systems can be used for plant genome engineering. Proof-of-concept experiments can be performed in plant leaf tissue by targeting DSBs to integrated reporter genes and endogenous loci. The technology then can be adapted for use in protoplasts and whole plants, and in viral-based delivery systems. Finally, multiplex genome engineering can be demonstrated by targeting DSBs to multiple sites within the same genome.

In general, the system and methods described herein include at least two components: the RNAs (crRNA and tracrRNA, or a single cr/tracrRNA hybrid) targeted to a particular sequence in a plant cell (e.g., in a plant genome, or in an extrachromosomal plasmid, such as a reporter), and a Cas9 endonuclease that can cleave the plant DNA at the target sequence. In some cases, a system also can include a nucleic acid containing a donor sequence targeted to a plant sequence. The endonuclease can to create targeted DNA double-strand breaks at the desired locus (or loci), and the plant cell can repair the double-strand break using the donor DNA sequence, thereby incorporating the modification stably into the plant genome.

The construct(s) containing the crRNA, tracrRNA, cr/tracrRNA hybrid, endonuclease coding sequence, and, where applicable, donor sequence, can be delivered to a plant cell using, for example, biolistic bombardment. Alternatively, the system components can be delivered using Agrobacterium-mediated transformation, insect vectors, grafting, or DNA abrasion, according to methods that are standard in the art, including those described herein. In some embodiments, the system components can be delivered in a viral vector (e.g., a vector from a DNA virus such as, without limitation, geminivirus, cabbage leaf curl virus, bean yellow dwarf virus, wheat dwarf virus, tomato leaf curl virus, maize streak virus, tobacco leaf curl virus, tomato golden mosaic virus, or Faba bean necrotic yellow virus, or a vector from an RNA virus such as, without limitation, a tobravirus (e.g., tobacco rattle virus, tobacco mosaic virus), potato virus X, or barley stripe mosaic virus.

After a plant is infected or transfected with an endonuclease encoding sequence and a crRNA and a tracrRNA, or a cr/tracrRNA hybrid (and, in some cases, a donor sequence), any suitable method can be used to determine whether GT or targeted mutagenesis has occurred at the target site. In some embodiments, a phenotypic change can indicate that a donor sequence has been integrated into the target site. Such is the case for transgenic plants encoding a defective GUS:NPTII reporter gene, for example. PCR-based methods also can be used to ascertain whether a genomic target site contains targeted mutations or donor sequence, and/or whether precise recombination has occurred at the 5′ and 3′ ends of the donor.

The invention will be further described in the following examples, which do not limit the scope of the invention described in the claims.

### EXAMPLES

### Example 1—Plasmids for Expressing CRISPR/Cas Components

To demonstrate functionality of the CRISPR/Cas systems for genome editing in plants, plasmids were constructed to encode Cas9, crRNA and tracrRNA, and the cr/tracrRNA hybrid. Plant codon-optimized Cas9 coding sequence was synthesized and cloned into a MultiSite Gateway entry plasmid. Additionally, crRNA and tracrRNA, or cr/tracrRNA hybrid, driven by the RNA polymerase III (PolIII) promoters AtU6-20 and At75L, were synthesized and cloned into a second MultiSite Gateway entry plasmid. To enable efficient reconstruction of the crRNA sequences (serving to redirect CRISPR/Cas-mediated DSBs), inverted BsaI restriction enzymes sites were inserted within the crRNA nucleotide sequence. By digesting with BsaI, target sequences can be efficiently cloned into the crRNA sequence using oligonucleotides. Entry plasmids for both Cas9 and the crRNA and tracrRNA, or the cr/tracrRNA hybrid, were recombined into pMDC32 (a standard T-DNA expression plasmid with a 2x35S promoter), pfZ19 (an estrogen-inducible T-DNA expression vector; Zuo et al., Plant J 2000, 24(2):265-273), and pNB121 (a geminivirus-replicon T-DNA vector).

### Example 2—CRISPR/Cas Activity in Somatic Plant Tissue

To demonstrate the capacity for CRISPR/Cas systems to function as SSNs, pMDC32 T-DNA plasmids are modified to encode both Cas9 and crRNA and tracrRNA, or cr/tracrRNA hybrid, sequences. Targeting RNA sequences (encoded by nucleotide sequence within the crRNA; responsible for directing Cas9 cleavage) are designed to be homologous to sequences within an integrated gus:nptII reporter gene or the endogenous SuRA and SuRB genes. T-DNA is delivered to Nicotiana tabacum leaf tissue by syringe infiltration with Agrobacterium tumefaciens. Five to seven days after infiltration, gus:nptII and SuRA/SuRB sequences are assessed for Cas9-mediated mutations using PCR-digest. The presence of mutations at the corresponding target sequences indicates functionality of CRISPR/Cas systems in plant leaf cells.

### Example 3—CRISPR/Cas Activity in Protoplasts

To further demonstrate the activity of CRISPR/Cas systems in plants, targeted mutagenesis of DNA sequence within Arabidopsis thaliana and Nicotiana tabacum protoplasts is assessed. Targeting crRNA sequences are redesigned to be homologous to sequences present within the endogenous ADH1 or TT4 genes (Arabidopsis), or the integrated gus:nptII reporter gene or SuRA/SuRB (Nicotiania). Protoplasts are isolated from Arabidopsis and Nicotiana leaf tissue and transfected with plasmids encoding Cas9 and the ADH1- or TT4-targeting crRNAs, or Cas9 and the gus:nptII- or SuRA/SuRB-targeting crRNA, respectively. Genomic DNA is extracted 5-7 days post transfection and assessed for mutations at the corresponding target sequences. In addition to targeting endogenous DNA sequences, the CRISPR/Cas system is assessed for the ability to cleave an extrachromosomal reporter plasmid. This reporter plasmid encodes a non-functional yellow fluorescent protein (YFP). YFP expression is disrupted by a direct repeat of internal coding sequence that flanks a target sequence for the Cas9/crRNA complex. The generation of targeted DSBs at the Cas9/crRNA target sequence results in recombination of the direct repeat sequences, thereby restoring YFP gene function. Transfection with plasmids encoding Cas9, crRNA, tracrRNA, or the cr/tracrRNA hybrid, and the YFP reporter is performed in both Arabidopsis and Nicotiana tabacum protoplasts. Restoration of YFP expression as a result of CRISPR/Cas nuclease activity is monitored by flow cytometry. Detecting mutations within ADH1, TT4, gus:nptII or SuRA/SuRB genes, or detecting YFP-expressing cells, indicates the functionality of CRISPR/Cas systems in plant protoplasts.

### Example 4—Multiplex Genome Engineering in Protoplasts Using CRISPR/Cas Systems

The ability of CRISPR/Cas systems to create multiple DSBs at different DNA sequences is assessed using plant protoplasts. To direct Cas9 nuclease activity to TT4, ADH1, and the extrachromosomal YFP reporter plasmid (within the same Arabidopsis protoplast), crRNA and tracrRNA or cr/tracrRNA hybrid plasmid is modified to express multiple crRNA targeting sequences. These sequences are designed to be homologous to sequences present within TT4, ADH1 and the YFP reporter plasmid. Following transfection with Cas9, crRNA,, tracrRNA, or the cr/tracrRNA hybrid, and YFP reporter plasmids into Arabidopsis protoplasts, YFP-expressing cells are quantified and isolated, and genomic DNA is extracted. Observing mutations within the ADH1 and TT4 genes in YFP-expressing cells suggests that CRISPR/Cas can facilitate multiplex genome engineering in Arabidopsis cells.

To demonstrate multiplex genome engineering in Nicotiana protoplasts, plasmids containing multiple crRNA are modified to encode sequences that are homologous to the integrated gus:nptII reporter gene, SuRA/SuRB, and the YFP reporter plasmid. Similar to the methods described in Arabidopsis protoplasts, Nicotiana protoplasts are transfected with Cas9, crRNA, tracrRNA, or the cr/tracrRNA hybrid, and YFP reporter plasmids. YFP-expressing cells are quantified and isolated, and genomic DNA is extracted. Observing mutations within the integrated gus:nptII reporter gene and SuRA/SuRB in YFP-expressing cells suggests that CRISPR/Cas can facilitate multiplex genome engineering in tobacco cells.

### Example 5—CRISPR/Cas Activity in Planta

To demonstrate CRISPR/Cas activity in planta, pFZ19 T-DNA is modified to encode both Cas9 and the crRNA and tracrRNA, or the cr/tracrRNA hybrid sequences. Target DNA sequences are present within the endogenous ADH1 or TT4 genes. The resulting T-DNA is integrated into the Arabidopsis thaliana genome by floral dip using Agrobacterium. Cas9 expression is induced in primary transgenic plants by direct exposure to estrogen. Genomic DNA from somatic leaf tissue is extracted and assessed for mutations at the corresponding genomic locus by PCR-digest. Observing mutations within the ADH1 or TT4 genes demonstrates CRISPR/Cas activity in planta. Alternatively, CRISPR/Cas activity can he assessed by screening T2 seeds (produced from induced T1 patents) for heterozygous or homozygous mutations at the corresponding genomic locus. Furthermore, the capacity for CRISPR/Cas to carry out multiplex genome engineering is assessed by modifying plasmids containing multiple crRNAs with homologous sequences to both ADH1 and TT4. The resulting T-DNA plasmid is integrated into the Arabidopsis genome, Cas9 expression is induced in primary transgenic plants, and CRISPR/Cas activity is assessed by evaluating the ADH1 and TT4 genes in both T1 and T2 plants. Observing mutations in both the ADH1 and TT4 genes suggests CRISPR/Cas can facilitate multiplex genome engineering in Arabidopsis plants.

### Example 6—Viral Delivery of CRISPR/Cas Components

Plant viruses can be effective vectors for delivery of heterologous nucleic acid sequence, such as for RNAi reagents or for expressing heterologous proteins. Useful plant viruses include both RNA viruses (e.g., tobacco mosaic virus, tobacco rattle virus, potato virus X, and barley stripe mosaic virus) and DNA viruses (e.g., cabbage leaf curl virus, bean yellow dwarf virus, wheat dwarf virus, tomato leaf curl virus, maize streak virus, tobacco leaf curl virus, tomato golden mosaic virus, and Faba bean necrotic yellow virus; Rybicki et al., Curr Top Microbiol Immunol, 2011; and Gleba et al., Curr Opin Biotechnol 2007, 134-141). Such plant viruses are modified for the delivery of CRISPR/Cas9 components. Proof-of-concept experiments are performed in Nicotiana tabacum leaf cells using DNA viruses (geminivirus replicons). To this end, crRNA sequences are modified to contain regions of homology to the integrated gus:nptII reporter gene or the endogenous SuRA/SuRB loci. The resulting plasmids are cloned into pNB121 (a T-DNA destination vector with cis-acting elements required for geminivirus replication (LSL T-DNA)) along with Cas9. Co-delivery of LSL T-DNA along with T-DNA encoding replicase protein (Rep; REP T-DNA) by Agrobacterium results in the replicational release of geminiviral replicons. The T-DNA is delivered to tobacco leaf tissue by syringe infiltration with Agrobacterium. Five to seven days after infiltration, gus:nptII and SuRA/SuRB sequences are assessed for Cas9-mediated mutations using PCR-digest. The presence of mutations at the corresponding target sequences indicates that plant viruses are effective vectors for delivery of CRISPR/Cas components.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A method comprising:
delivering a construct containing nucleic acid sequences encoding a Cas9 endonuclease molecule of Streptococcus pyogenes and RNA to a plant cell, wherein the RNA includes:
a Clustered Regularly Interspersed Short Palindromic Repeats RNA (crRNA) and a trans-activating RNA (tracrRNA), the crRNA and tracrRNA being targeted to a target sequence that is endogenous to the plant cell; and
culturing the plant cell with the delivered construct under conditions to induce:
targeting of the target sequence by the RNA; and
generating a double stranded break at or near the target sequence to which the crRNA and tracrRNA are targeted by the Cas9 endonuclease molecule.

3. The method of claim 2, wherein the plant cell is a dicotyledonous plant cell.

4. The method of claim 2, wherein the plant cell is a monocotyledonous plant cell.

5. The method of claim 2, wherein the crRNA and tracrRNA include a chimeric cr/tracrRNA hybrid and the method further includes detecting the presence of a mutation at or near the sequence to which the cr/tracrRNA hybrid is targeted.

6. The method of claim 2, wherein the Cas9 endonuclease molecule and the RNA are encoded as a Cas9/RNA complex and targeting of the target sequence includes directing the Cas9 protein/RNA complex to the target sequence.

7. The method of claim 2, wherein generating the double stranded break includes generating a double stranded DNA break at or near target sequence by the Cas9 endonuclease molecule, wherein the nucleic acid sequence encoding the Cas9 endonuclease molecule is operably linked to a promoter that is constitutive, cell specific, inducible, or activated by alternative splicing of a suicide exon.

8. The method of claim 2, wherein delivering the construct includes delivering to the plant cell via a virus or bacterium strain.

9. The method of claim 8, wherein the virus is a DNA virus or an RNA virus.

10. The method of claim 9, wherein the DNA virus is selected from the group consisting of geminivirus, cabbage leaf curl virus, bean yellow dwarf virus, wheat dwarf virus, tomato leaf curl virus, maize streak virus, tobacco leaf curl virus, tomato golden mosaic virus, and Faba bean necrotic yellow virus.

11. The method of claim 9, wherein the RNA virus is selected from the group consisting of tobraviruses, potato virus X, and barley stripe mosaic virus.

12. The method of claim 7, wherein delivering the construct includes a T-DNA delivery via Agrobacterium or Ensifer.

13. The method of claim 1, wherein delivering the construct includes delivering to protoplasts.

14. The method of claim 1, wherein:
the plant cell is selected from the group consisting of a tomato plant cell, a soybean plant cell, a tobacco plant cell, or a potato plant cell;
the nucleic acid sequence encoding the RNA is operably linked to an RNA polymerase III (PolIII) promoter selected from AtU6-20 and At75L; and
the nucleic acid sequence encoding the Cas9 endonuclease molecule is operably linked to a promoter that is constitutive, cell specific, inducible, or activated by alternative splicing of a suicide exon.

15. A construct containing nucleotide acid sequences encoding:
a Cas9 endonuclease molecule of Streptococcus pyogenes; and
RNA including:
a crRNA; and
a tracrRNA, wherein the crRNA and tracrRNA are targeted to a target sequence that is endogenous to a plant genome and the Cas9 endonuclease molecule induces a double stranded break at or near the target sequence to which the crRNA and tracrRNA are targeted.

16. The construct of claim 14, wherein the crRNA and tracrRNA include a chimeric cr/tracrRNA hybrid.

17. The construct of claim 14, wherein the Cas9 endonuclease molecule and the RNA are encoded as a Cas9/RNA complex.

18. The construct of claim 14, wherein:
the Cas9 endonuclease molecule is operably linked to a promoter that is constitutive, cell specific, inducible, or activated by alternative splicing of a suicide exon; and
the RNA is operably linked to an RNA polymerase III (PolIII) promoter selected from AtU6-20 and At75L.

19. The construct of claim 14, wherein the plant genome and the target sequence is associated with a dicotyledonous plant or a monocotyledonous plant.
